(19) United States
(12) Patent Application Publication
(10) Pub. No.: US 2022/0048178 A1
(43) Pub. Date: Feb. 17, 2022

(54) PUSH-PULL PRESS-CONTACT APPARATUS AND ANTISEPTIC CASE

(71) Applicant: SUMEC HARDWARE & TOOLS CO., LTD., Nanjing (CN)

(72) Inventors: Lin-Miao CHEN, NANJING (CN); Kai LIU, NANJING (CN); Yan-Xiang ZHAO, NANJING (CN); Yi-Gang TANG, NANJING (CN)

(21) Appl. No.: 17/011,666

(22) Filed: Sep. 3, 2020

(30) Foreign Application Priority Data
Aug. 14, 2020 (CN) 202010817395.X

(51) Int. Cl. B25F 1/00; E05B 1/00; F16B 45/00; B25G 1/10

(52) U.S. Cl. CPC B25F 1/00; E05B 1/0069; B25B 33/00; B25G 1/102; F16B 45/00

(57) ABSTRACT

A push-pull press-contact apparatus includes holding member capable of being held with fingers; push-pull member connected to the holding member and having push portion, stop portion and pull portion sequentially connected; and pressing member connected to the pull portion from front. An antiseptic case includes case member having mounting space; alcoholic sponge mounted in the mounting space and having slot insertable by push-pull member and pressing member; and lid for covering the case member. Another antiseptic case includes case member having mounting space; alcoholic sponge mounted in the mounting space and having slot insertable by push-pull member and pressing member of push-pull press-contact apparatus; and lid for covering the case member.

### BACKGROUND OF THE INVENTION

### 1. Technical Field

The present disclosure relates to wearable apparatuses and, more particularly, to a push-pull press-contact apparatus and an antiseptic case.

### 2. Description of the Related Art

Conventional wearable apparatuses for use in push-pull press-contact are typically disadvantaged by incomplete push-pull press-contact functionality to the detriment of ease of use. Therefore, conventional push-pull press-contact apparatuses still have room for improvement. Furthermore, conventional push-pull press-contact apparatuses lack dedicated cases and thus are likely to come into contact with users' hands or clothes and even get lost, thereby having room for improvement.

### BRIEF SUMMARY OF THE INVENTION

It is an objective of the present disclosure to provide a push-pull press-contact apparatus for providing a push-pull press-contact structure which is easy to use and effective.

Another objective of the present disclosure is to provide an antiseptic case which conveniently contains and sterilizes the push-pull press-contact apparatus.

In order to achieve the above and other objectives, the present disclosure provides a push-pull press-contact apparatus, comprising a holding member capable of being held with fingers; a push-pull member connected to the holding member and having a push portion, a stop portion and a pull portion which are sequentially connected, wherein the push-pull member defines a receiving space and an opening; and a pressing member connected to the pull portion of the push-pull member from front.

The holding member, push-pull member and pressing member jointly provide a push-pull press-contact structure which is easy to use and effective, thereby achieving the objective of the present disclosure.

Preferably, the stop portion has a bevel or rounded corner relative to the push portion and the pull portion.

Preferably, the push-pull member has a hook portion connected to the pull portion from behind.

Preferably, the push-pull press-contact apparatus further comprises a head member connected to the holding member from above.

Preferably, the pressing member is covered with copper.

Preferably, the holding member connects to the push portion of the push-pull member and thus forms a finger hole penetrable by fingers.

In order to achieve the above and other objectives, the present disclosure provides an antiseptic case for containing the push-pull press-contact apparatus. The antiseptic case comprises a case member having a mounting space; an alcoholic sponge mounted in the mounting space of the case member and having a slot which the push-pull member and pressing member of the push-pull press-contact apparatus are inserted into; and a lid for covering the case member.

The case member, alcoholic sponge and lid together conveniently contain and sterilize the push-pull press-contact apparatus, thereby achieving the objective of the present disclosure.

Preferably, the lid is pivotally, openly connected to the case member.

In order to achieve the above and other objectives, the present disclosure provides an antiseptic case, comprising a case member having a mounting space; an alcoholic sponge mounted in the mounting space of the case member and having a slot which a push-pull member and a pressing member of a push-pull press-contact apparatus are inserted into; and a lid for covering the case member.

The case member, alcoholic sponge and lid together conveniently contain and sterilize the push-pull press-contact apparatus, thereby achieving the objective of the present disclosure.

Preferably, the lid is pivotally, openly connected to the case member.

### BRIEF DESCRIPTION OF THE SEVERAL VIEWS OF THE DRAWINGS

FIG. 1 is a perspective view of a push-pull press-contact apparatus according to a preferred embodiment of the present disclosure.

FIG. 2 is a front view of the push-pull press-contact apparatus according to a preferred embodiment of the present disclosure.

FIG. 3 is a schematic view of how to operate the push-pull press-contact apparatus according to a preferred embodiment of the present disclosure.

FIG. 4 is another schematic view of how to operate the push-pull press-contact apparatus according to a preferred embodiment of the present disclosure.

FIG. 5 is yet another schematic view of how to operate the push-pull press-contact apparatus according to a preferred embodiment of the present disclosure.

FIG. 6 is still yet another schematic view of how to operate the push-pull press-contact apparatus according to a preferred embodiment of the present disclosure.

FIG. 7 is an assembled perspective view of the push-pull press-contact apparatus and an antiseptic case according to a preferred embodiment of the present disclosure.

FIG. 8 is a schematic view of how to open a lid of the antiseptic case according to a preferred embodiment of the present disclosure.

FIG. 9 is a cross-sectional view taken along line 9-9 of FIG. 7.

FIG. 10 is a cross-sectional view taken along line 10-10 of FIG. 9.

FIG. 11 is an exploded view of the push-pull press-contact apparatus and the antiseptic case according to a preferred embodiment of the present disclosure.

### DETAILED DESCRIPTION OF THE INVENTION

The present disclosure is hereunder illustrated with embodiments, depicted with accompanying drawings, and described below.

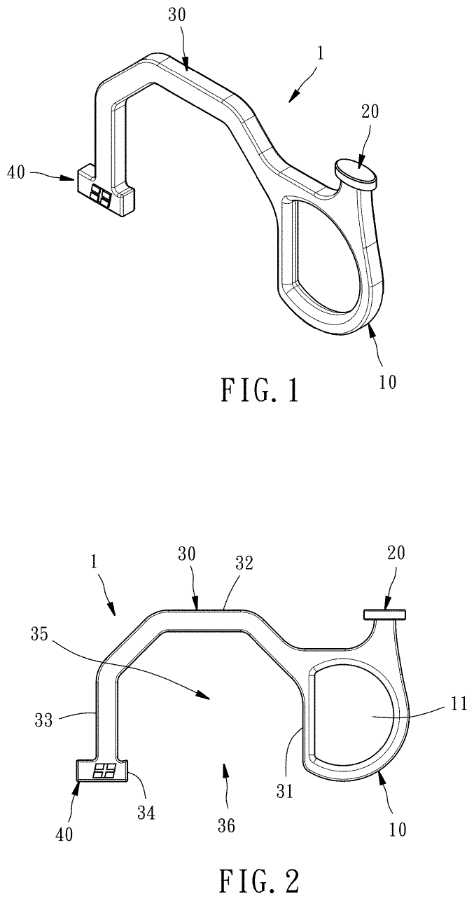

Referring to FIG. 1 and FIG. 2, the present disclosure, in a preferred embodiment thereof, provides a push-pull press-contact apparatus 1 comprising a holding member 10, a head member 20, a push-pull member 30, and a pressing member 40.

The holding member 10 can be held with fingers.

The head member 20 is connected to the holding member 10 from above, pressed with thumb, and supported by the other fingers.

The push-pull member 30 is connected to the holding member 10 and comprises sequentially-connected elements as follows: a push portion 31, a stop portion 32, a pull portion 33, and a hook portion 34 connected to the pull portion 33 from behind. Therefore, the push-pull member 30 defines a receiving space 35 and an opening 36.

The stop portion 32 has a bevel or rounded corner relative to the push portion 31 and pull portion 33.

The holding member 10 connects to the push portion 31 of the push-pull member 30 and thus forms a finger hole 11 penetrable by fingers.

The pressing member 40 is connected to the pull portion 33 of the push-pull member 30 from front.

The pressing member 40 is covered with copper.

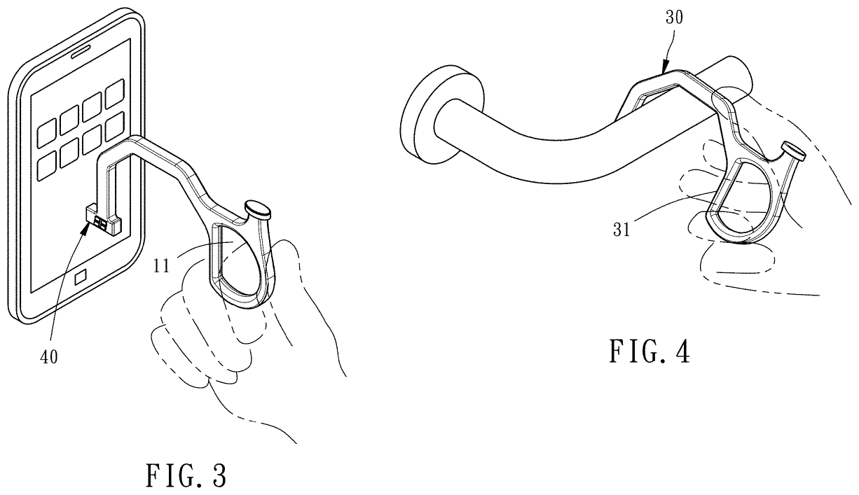

FIG. 3 is a schematic view of how to operate a touch panel with the pressing member 40 according to a preferred embodiment of the present disclosure.

FIG. 4 is another schematic view of how to push or pull a small-caliber door handle with the push-pull member 30 according to a preferred embodiment of the present disclosure.

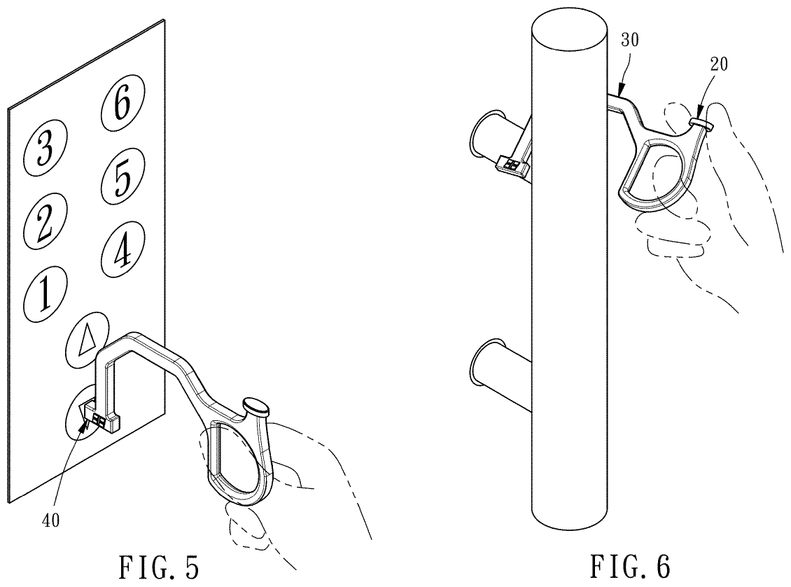

FIG. 5 is yet another schematic view of how to press a button with the pressing member 40 according to a preferred embodiment of the present disclosure.

FIG. 6 is still yet another schematic view of how to push or pull a large-caliber door handle with the push-pull member 30 according to a preferred embodiment of the present disclosure.

In this embodiment, the holding member 10, push-pull member 30 and pressing member 40 jointly provide a push-pull press-contact structure which is easy to use and effective. Furthermore, the pressing member 40 is covered with copper to eliminate bacteria and virus quickly.

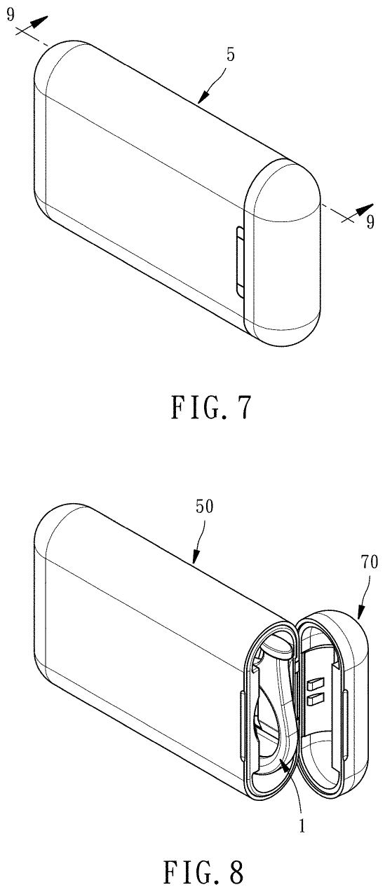

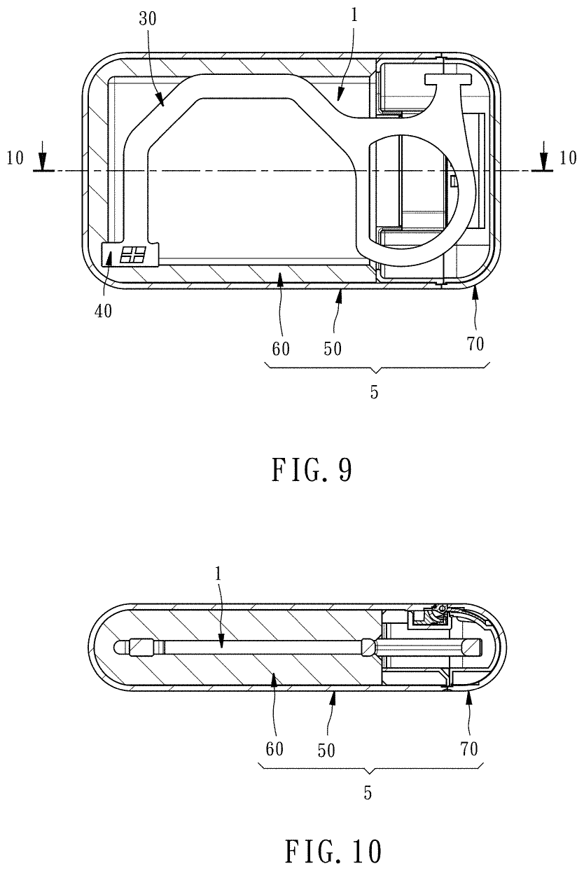

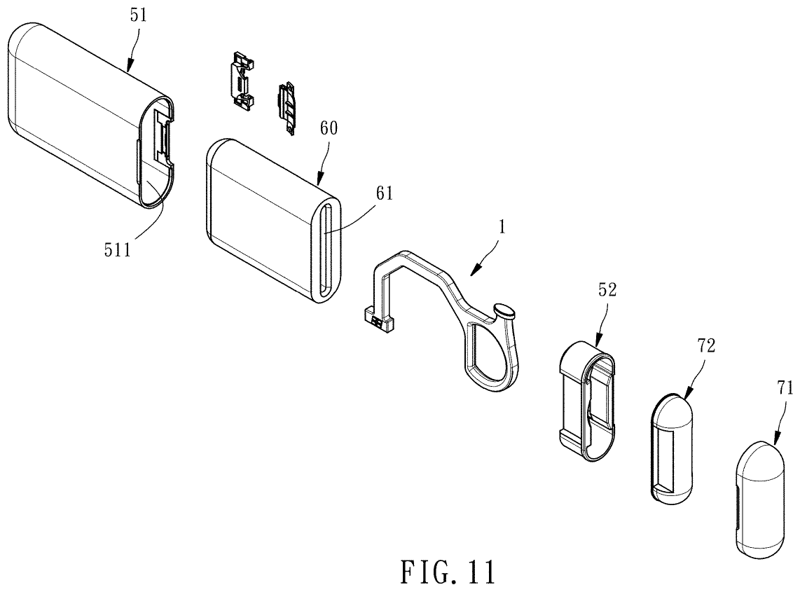

Referring to FIG. 7 through FIG. 11, the present disclosure provides an antiseptic case 5 for containing the push-pull press-contact apparatus 1. The antiseptic case 5 comprises a case member 50, an alcoholic sponge 60 and a lid 70.

The case member 50 comprises a case body exterior 51 and a case body opening frame 52. The case body exterior 51 defines therein a mounting space 511. The case body opening frame 52 is disposed at the opening of the mounting space 511 of the case body exterior 51.

The alcoholic sponge 60 is mounted in the mounting space 511 of the case member 50. The alcoholic sponge 60 has a slot 61 which the push-pull member 30 and pressing member 40 of the push-pull press-contact apparatus 1 are inserted into.

The lid 70 is pivotally, openly connected to the case member 50. The lid 70 has a lid exterior 71 and a lid interior 72 and is connected to the case member 50 by a pivotal connection means.

The case member 50, alcoholic sponge 60 and lid 70 together conveniently contain and sterilize the push-pull press-contact apparatus 1, thereby achieving the objective of the present disclosure.

However, the application of the antiseptic case 5 of the present disclosure is not restricted to the push-pull press-contact apparatus 1. In this regard, the antiseptic case 5 comprises a case member 50, an alcoholic sponge 60 and a lid 70.

The case member 50 comprises a case body exterior 51 and a case body opening frame 52. The case body exterior 51 defines therein a mounting space 511. The case body opening frame 52 is disposed at the opening of the mounting space 511 of the case body exterior 51.

The alcoholic sponge 60 is mounted in the mounting space 511 of the case member 50. The alcoholic sponge 60 has a slot 61 which a push-pull member 30 and a pressing member 40 of a push-pull press-contact apparatus 1 are inserted into.

The lid 70 is pivotally, openly connected to the case member 50. The lid 70 has a lid exterior 71 and a lid interior 72 and is connected to the case member 50 by a pivotal connection means.

The case member 50, alcoholic sponge 60 and lid 70 together conveniently contain and sterilize the push-pull press-contact apparatus 1, thereby achieving the objective of the present disclosure.

The present disclosure further provides a combination of the push-pull press-contact apparatus 1 and the antiseptic case 5.

What is claimed is:

1. A push-pull press-contact apparatus (1), comprising:
a holding member (10) capable of being held with fingers;
a push-pull member (30) connected to the holding member (10) and having a push portion (31), a stop portion (32) and a pull portion (33) which are sequentially connected, wherein the push-pull. member (30) defines a receiving space (35) and an opening (36); and
a pressing member (40) connected to the pull portion (33) of the push-pull member (30) from front.

2. The push-pull press-contact apparatus (1) of claim 1, wherein the stop portion (32) has a bevel or rounded corner relative to the push portion (31) and the pull portion (33).

3. The push-pull press-contact apparatus (1) of claim 1, wherein the push-pull member (30) further has a hook portion (34) connected to the pull portion (33) from behind.

4. The push-pull press-contact apparatus (1) of claim 1, further comprising a head member (20) connected to the holding member (10) from above.

5. The push-pull press-contact apparatus (1) of claim 1, wherein the pressing member (40) is covered with copper.

6. The push-pull press-contact apparatus (1) of claim 1, wherein the holding member (10) connects to the push portion (31) of the push-pull member (30) and thus forms a finger hole (11) penetrable by fingers.

7. An antiseptic case (5) for containing the push-pull press-contact apparatus (1) of claim 1, comprising:
a case member (50) having a mounting space (511);
an alcoholic sponge (60) mounted in the mounting space (511) of the case member (50) and having a slot (61) which the push-pull member (30) and the pressing member (40) of the push-pull press-contact apparatus (1) are inserted into; and
a lid (70) for covering the case member (50).

8. The antiseptic case (5) of claim 7, wherein the lid (70) is pivotally, openly connected to the case member (50).

9. An antiseptic case (5), comprising:
a case member (50) having a mounting space (511);
an alcoholic sponge (60) mounted in the mounting space (511) of the case member (50) and having a slot (61) which a push-pull member (30) and a pressing member (40) of a push-pull press-contact apparatus (1) are inserted into; and
a lid (70) for covering the case member (50).

10. The antiseptic case (5) of claim 9, wherein the lid (70) is pivotally, openly connected to the case member (50).
